(19) United States
(12) Patent Application Publication
(10) Pub. No.: US 2021/0282653 A1
(43) Pub. Date: Sep. 16, 2021

(54) BIOMETRIC INFORMATION MEASURING APPARATUS

(71) Applicant: SONY CORPORATION, TOKYO (JP)

(72) Inventor: ATSUSHI ITO, TOKYO (JP)

(21) Appl. No.: 17/250,429

(22) PCT Filed: Jun. 17, 2019
(86) PCT No.: PCT/JP2019/023792

§ 371 Date: Jan. 20, 2021

(30) Foreign Application Priority Data
Jul. 30, 2018 (JP) 2018-142513

(51) Int. Cl. A61B 5/026; A61B 5/00

(52) U.S. Cl. CPC A61B 5/0261; A61B 2560/04; A61B 5/0059

(57) ABSTRACT

The present technology provides a biometric information measuring apparatus capable of reducing noise caused by a body movement. That is, the present technology provides a biometric information measuring apparatus including: a housing that accommodates therein a light emitting part that emits coherent light and a light receiving part and has a lid part through which the coherent light transmits; a light transmitting layer located outside the lid part and through which the coherent light transmits; and a light shielding film that prevents the coherent light from being reflected by the light transmitting layer and reaching the light receiving part.

### TECHNICAL FIELD

The present technology relates to a biometric information measuring apparatus.

### BACKGROUND ART

A laser Doppler blood flow meter is known as an apparatus for non-invasively measuring the blood flow velocity under the skin by irradiating the skin with coherent light and analyzing the backward scattered light (for example, Patent Documents 1 and 2). A blood flow sensor mounted on a blood flow meter is often manufactured as one package containing a laser diode and a photodiode. The package is arranged on a circuit board together with an electric processing circuit such as an amplifier, and is mounted on a blood flow meter. A technology is known in which, in order to protect a circuit board from foreign substances such as sweat and dust, a cover part including a translucent member is provided on a blood flow meter (for example, Patent Document 3).

### CITATION LIST

### Patent Document

Patent Document 1: Japanese Patent Application Laid-Open No. 2004-229920
Patent Document 2: International Publication No. 2009/139029
Patent Document 3: International Publication No. 2016/067359

### SUMMARY OF THE INVENTION

### Problems to be Solved by the Invention

The present inventor has found that, if a translucent member that prevents foreign substances from entering as described in Patent Document 3 is provided, in a case where a body movement occurs during measurement, large noise is generated in the measured value.

Therefore, the main purpose of the present technology is to provide a biometric information measuring apparatus capable of reducing noise caused by a body movement.

### Solutions to Problems

That is, the present technology provides a biometric information measuring apparatus including:

a housing that accommodates therein a light emitting part that emits coherent light and a light receiving part and has a lid part through which the coherent light transmits;

a light transmitting layer located outside the lid part and through which the coherent light transmits; and

a light shielding film that prevents the coherent light from being reflected by the light transmitting layer and reaching the light receiving part. The biometric information measuring apparatus may be configured such that

the light shielding film is provided on at least one of an inner surface of the lid part or an outer surface of the lid part, and has a light emitting side opening part facing the light emitting part and a light receiving side opening part facing the light receiving part, and

in a case where a distance from a light emitting center of the light emitting part to a reflecting surface of the light transmitting layer on which the coherent light is reflected is D₁,

a distance from a light shielding surface of the light shielding film to the reflecting surface is D₂,

a distance from a light receiving surface of the light receiving part to the reflecting surface is D₃,

a distance from a position corresponding to the light emitting center of the light emitting part in the light emitting side opening part to a proximal end of the light receiving side opening part is L₁, and

a distance from the light emitting center of the light emitting part to a distal end of the light receiving part is L₂, the Equation (1) described below is satisfied.

$$\begin{matrix}[ Math . 1 ] & \\ \frac{{L}_{1}}{{D}_{1} + {D}_{2}} \geq \frac{{L}_{2}}{{D}_{1} + {D}_{3}} & ( 1 )\end{matrix}$$

The biometric information measuring apparatus may be configured such that

the light shielding film is provided on at least one of an inner surface of the lid part or outer surface of the lid part, and has a light emitting side opening part facing the light emitting part and a light receiving side opening part facing the light receiving part, and

in a case where a distance from a light emitting center of the light emitting part to a reflecting surface of the light transmitting layer on which the coherent light can be reflected is D₁,

a distance from a light shielding surface of the light shielding film to the reflecting surface is D₂,

a distance from a light receiving surface of the light receiving part to the reflecting surface is D₃,

a distance from the light emitting center of the light emitting part to a distal end of the light receiving part is L₂, and

a distance from a position corresponding to the light emitting center of the light emitting part in the light emitting side opening part to an end of a center direction of the light receiving side opening part of the light emitting side opening part is L₃,

the Equation (2) described below is satisfied.

$$\begin{matrix}[ Math . 2 ] & \\ \frac{{L}_{3}}{{D}_{1} - {D}_{2}} \leq \frac{{L}_{2}}{{D}_{1} + {D}_{3}} & ( 2 )\end{matrix}$$

The biometric information measuring apparatus may be configured such that

the light shielding film is provided on at least one of an outer surface of the lid part, an inner surface of the light transmitting layer, or an outer surface of the light transmitting layer, and

in a case where a distance from a light emitting center of the light emitting part to a plane including a light shielding surface of the light shielding film is D₄,

a distance from a light receiving surface of the light receiving part to the plane including the light shielding surface of the light shielding film is D₅,

a distance from the light emitting center of the light emitting part to a light receiving surface center of the light receiving part is L₄,

a length of the light shielding film in a width direction when a direction connecting the light emitting center of the light emitting part and the light receiving surface center of the light receiving part is the width direction is W₁, and

a length of the light receiving part in the width direction is W₂,

the Equation (3) described below is satisfied.

$$\begin{matrix}[ Math . 3 ] & \\ {W}_{1} \geq \frac{{D}_{4} - {D}_{5}}{{D}_{4} + {D}_{5}} {L}_{4} + \frac{{D}_{4}}{{D}_{4} + {D}_{5}} {W}_{2} & ( 3 )\end{matrix}$$

The housing may accommodate two or more of the light receiving part therein.

The housing may accommodate three or more of the light receiving part therein, and the three or more of the light receiving part may be arranged at unequal intervals.

The biometric information measuring apparatus may include a connection pad electrically connected to the light emitting part at a position corresponding to a portion between the light receiving parts.

The biometric information measuring apparatus may include an electrical component at a position corresponding to a portion between the light receiving parts.

According to the present technology, it is possible to reduce noise caused by a body movement in an apparatus for measuring biological information. Note that, the effect of the present technology is not necessarily limited to the effect described herein, and any of the effects described in this specification may be used.

### BRIEF DESCRIPTION OF DRAWINGS

FIG. 1 is a schematic cross-sectional view showing a part of a biometric information measuring apparatus 10 of the present technology.

FIG. 2 is a schematic cross-sectional view showing a part of a conventional laser Doppler blood flow meter 910.

FIG. 3 is a schematic cross-sectional view showing a part of the biometric information measuring apparatus 10 according to a first embodiment.

FIG. 4 is a schematic cross-sectional view showing a part of a biometric information measuring apparatus 11 according to a second embodiment.

FIG. 5 is a schematic cross-sectional view showing a part of a biometric information measuring apparatus 12 according to a third embodiment.

FIG. 6 is a schematic view showing a part of a biometric information measuring apparatus 13 according to a fourth embodiment.

FIG. 7 is a schematic plan view showing a part of a biometric information measuring apparatus 14 according to a fifth embodiment.

FIG. 8 is a schematic plan view showing a part of a biometric information measuring apparatus 15 according to a sixth embodiment.

FIG. 9 is a schematic view showing a substrate 90 on which a housing 40 shown in FIG. 8 is mounted.

### MODE FOR CARRYING OUT THE INVENTION

Hereinafter, suitable modes for carrying out the present technology will be described with reference to the drawings. Note that the embodiments described below are typical embodiments of the present technology, and the scope of the present technology should not be construed narrowly. The description will be given in the following order.

1. Overall configuration of biometric information measuring apparatus

2. Operation of biometric information measuring apparatus

3. Explanation of cause of noise generated when body movement occurs

4. First embodiment (configuration example of light shielding film)

5. Second embodiment (configuration example of light shielding film)

6. Third embodiment (configuration example of light shielding film)

7. Fourth embodiment (configuration example including two or more light receiving parts)

8. Fifth embodiment (configuration example in which light receiving parts are arranged at unequal intervals)

9. Sixth embodiment (configuration example including electrical components)

### 1. Overall Configuration of Biometric Information Measuring Apparatus

The overall configuration of a biometric information measuring apparatus according to the present technology will be described.

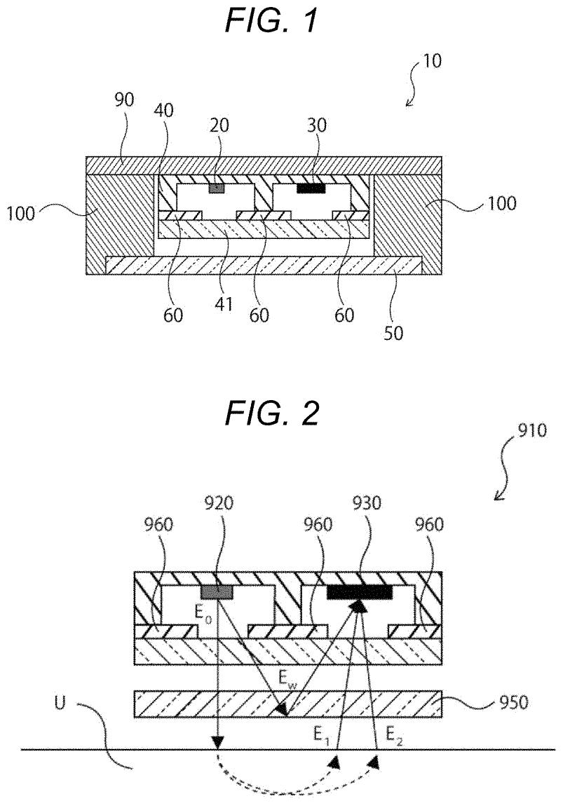

FIG. 1 is a schematic cross-sectional view showing a part of a biometric information measuring apparatus 10 of the present technology. The biometric information measuring apparatus 10 will be described below assuming that the biometric information measuring apparatus 10 is a laser Doppler blood flow meter as an example.

The biometric information measuring apparatus 10 includes a housing 40, a light transmitting layer 50, a light shielding film 60, a substrate 90, and a main body part 100.

The housing 40 accommodates therein a light emitting part 20 that emits coherent light and a light receiving part 30, and has a lid part 41. The housing 40, the light emitting part 20, the light receiving part 30, and the lid part 41 correspond to members included in a blood flow sensor. Examples of the light emitting part 20 include a laser diode. Examples of the light receiving part 30 include a photodiode and a phototransistor. The lid part 41 is formed by a translucent member through which coherent light transmits. The translucent member is only required to be a member through which at least the coherent light emitted from the light emitting part 20 can transmit, and light other than the coherent light may not be able to transmit through the translucent member. For example, in a case where infrared light is used as the light source of the light emitting part 20, the lid part 41 may be formed by a member through which only infrared light transmits and which absorbs light other than infrared light. From the viewpoint of preventing unnecessary light from entering the light receiving part 30, the lid part 41 is preferably formed by a member through which light other than the coherent light emitted from the light emitting part 20 does not transmit. Examples of the material of the translucent member include glass and resin.

The light transmitting layer 50 is formed by a translucent member through which coherent light transmits. As similar to the translucent member used in the lid part 41, the translucent member used for the light transmitting layer 50 is only required to be a member through which at least coherent light can transmit. The translucent member used for the light transmitting layer 50 may be the same as or different from the translucent member used for the lid part 41. The light transmitting layer 50 has a function of protecting a circuit board or the like (not shown) arranged inside the biometric information measuring apparatus 10 from foreign substances such as sweat and dust. Therefore, it is possible to further improve the mechanical reliability of the biometric information measuring apparatus 10.

The light shielding film 60 has a function of preventing the coherent light emitted from the light emitting part 20 from being reflected by the light transmitting layer 50 and reaching the light receiving part 30. The configuration of the light shielding film 60 will be described in detail later.

The housing 40 is mounted on the substrate 90. The substrate 90 and the light transmitting layer 50 are fixed by a main body part 100 so that the lid part 41 of the housing 40 and the light transmitting layer 50 face each other. The housing 40 is arranged in a space surrounded by the substrate 90, the light transmitting layer 50, and the main body part 100.

### 2. Operation of Biometric Information Measuring Apparatus

When the biometric information measuring apparatus 10 is used, coherent light is emitted from the light emitting part 20 toward the skin in a state where the light transmitting layer 50 is in contact with the user's skin. The emitted coherent light passes through the lid part 41, the light transmitting layer 50, and the skin surface, and reaches the blood vessels under the skin. The light scattered by the blood cells contained in the blood flowing through the blood vessels is detected by the light receiving part 30. The biometric information measuring apparatus 10 obtains biological information such as blood flow velocity by analyzing the detected scattered light.

### 3. Explanation of Cause of Noise Generated when Body Movement Occurs

FIG. 2 is a schematic cross-sectional view showing a part of a conventional laser Doppler blood flow meter 910. The cause of noise generated when a body movement occurs will be described with reference to FIG. 2.

The laser Doppler blood flow meter 910 includes a light emitting part 920, a light receiving part 930, a cover part 950 including a translucent member, and a shielding layer 960. The light emitting part 920 emits coherent light toward a test site of a user U. The cover part 950 has a function of protecting a circuit board or the like (not shown) arranged inside the laser Doppler blood flow meter 910 from foreign substances. Note that the shielding layer 960 shown in FIG. 2 is a component adopted in the prior art, and does not have the same function as the light shielding film 60 of the present technology shown in FIG. 1.

First, a case where the cover part 950 including a translucent member is not provided will be described. An electric field of the coherent light emitted from the light emitting part 920 is set to E₀, and a frequency at that time is set to co. An electric field of light returning via the blood vessels under the skin, which is the test site of user U, includes both light E₁ with a frequency ω that is not Doppler-shifted by blood and light E₂ with a frequency ω+Δω that is Doppler-shifted by blood, and the light E₁ and the light E₂ are superposed on the light receiving part 930. The electric field E_{PD} of the light appearing on the light receiving part 930 is expressed by the Equation (4) described below.

[Math. 4]

E_{PD}=E₁(ω)+E₂(ω+Δω) (4)

When a body movement occurs under the conditions described above, light E₁ and light E₂ change due to the influence of changes in the measurement position or the like, and the electric field E_{PD} of light is expressed by the Equation (5) described below.

[Math. 5]

E_{PD}= [symbol] (ω)+ [symbol] (ω+Δω) (5)

In this case, since the signal observed in the light receiving part 930 is the absolute value of the electric field, the signal P_{PD} is expressed by the Equation (6) described below.

[Math. 6]

P_{PD}= [symbol] ²+ [symbol] ²+2 [symbol] (6)

Here, the last term of the Equation (6) described above is an interference term that appears in the light receiving part 930 called a beat signal. The laser Doppler blood flow meter 910 calculates the blood flow velocity and the like by analyzing the frequency of this beat signal. Since the beat signal shown in the Equation (6) described above includes the frequency of light affected by a body movement, the values (blood flow velocity and the like) obtained by analyzing this beat signal include a value including body movement noise.

Next, a case where the laser Doppler blood flow meter 910 includes the cover part 950 will be described. When a body movement occurs, the test site (blood vessel under the skin) of the user U and the cover part 950 move at different speeds, so that the coherent light emitted from the light emitting part 920 passes through two target objects that move differently, that is, the test site and the cover part 950. Therefore, reflected light E_{w} from an optical interface (hereinafter, also simply referred to as an interface) of the cover part 950 is added to the light E₁ and the light E₂ that have passed through the test site. As a result, the electric field E_{PD} of the light appearing on the light receiving part 930 and the signal P_{PD} observed in the light receiving part 930 are expressed by the Equations (7) and (8) described below, respectively.

[Math. 7]

E_{PD}=E₁(ω)+E₂(ω+Δω)+E_{w}(ω) (7)

[Math. 8]

P_{PD}= [symbol] ²+ [symbol] ²+ [symbol] ²+2 [symbol] +2 [symbol] +2 [symbol] (8)

Two terms are added to the Equation (8) of a case where the cover part 950 is provided, as compared to the Equation (6) of a case where the cover part 950 is not provided. As a result, noise caused by the cover part 950 is further superimposed on the beat signal observed in the light receiving part 930, and when a body movement occurs, the noise is significantly deteriorated by the cover part 950. In particular, the outer surface of the cover part 950 is in contact with the skin of user U, so that it is easily scratched or soiled. When these discontinuous structures are formed at the interface, the portions become bright spots and shine, which greatly affects the results observed in the light receiving part 930.

Note that the light receiving part 930 also detects light (not shown) reflected on the user's skin surface. The light reflected on the skin surface is contained in the light E₁ having a frequency ω that is not Doppler-shifted by blood.

As described above, the present inventor has found that, in a biometric information measuring apparatus provided with a translucent member that uses coherent light and protects the inside, when a body movement occurs, the coherent light is reflected at the interface of the translucent member and reaches the light receiving part, and this causes noise. Note that such a problem does not occur in a device (for example, a conventional pulse wave meter or the like) for measuring a change in the amount of light returning to the light receiving part using non-coherent light such as an LED as a light source.

As a result of diligent studies to reduce the above-mentioned noise caused by a body movement, the present inventor has found that the noise described above can be reduced by providing a specific light shielding film, and has completed the present invention. Hereinafter, the biometric information measuring apparatus of the present technology will be described in detail.

### 4. First Embodiment

A biometric information measuring apparatus according to a first embodiment of the present technology will be described.

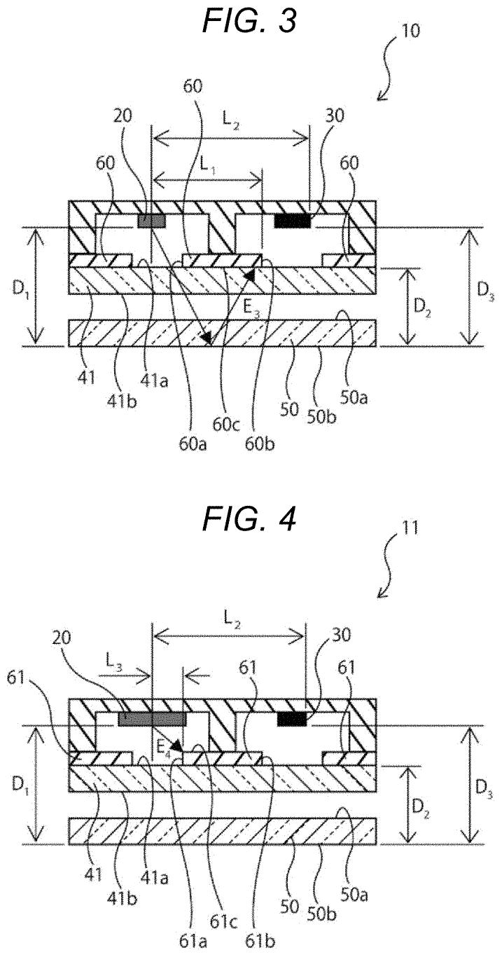

FIG. 3 is a schematic cross-sectional view showing a part of the biometric information measuring apparatus 10 according to the first embodiment. In FIG. 3, the substrate 90 and the main body part 100 shown in FIG. 1 are not shown. Furthermore, in FIG. 3, similar components to those shown in FIG. 1 are designated by the same reference numerals, and their description will be omitted as appropriate.

The light shielding film 60 included in the biometric information measuring apparatus 10 is provided on an inner surface 41a of the lid part 41, and has a light emitting side opening part 60a facing the light emitting part 20 and a light receiving side opening part 60b facing the light receiving part 30. FIG. 3 shows a cross section of a surface passing through the light emitting center of the light emitting part 20, the light receiving surface center of the light receiving part 30, the center of the light emitting side opening part 60a, and the center of the light receiving side opening part 60b.

The light shielding film 60 of the biometric information measuring apparatus 10 of the present embodiment prevents the coherent light emitted from the light emitting part 20 from being reflected by the light transmitting layer 50 and reaching the light receiving part 30. Specifically, as shown in FIG. 3, the reflected light E₃ from the interface of the light transmitting layer 50 is shielded to prevent the reflected light E₃ from reaching the light receiving part 30. Therefore, it is possible to reduce noise caused by the light reflected at the interface of the light transmitting layer 50 when a body movement occurs.

The biometric information measuring apparatus 10 of the present embodiment satisfies the Equation (1) described below in a case where D₁, D₂, D₃, L₁, and L₂ are defined as follows.

$$\begin{matrix}[ Math . 9 ] & \\ \frac{{L}_{1}}{{D}_{1} + {D}_{2}} \geq \frac{{L}_{2}}{{D}_{1} + {D}_{3}} & ( 1 )\end{matrix}$$

In the Equation (1) described above,

D₁ is a distance from the light emitting center of the light emitting part 20 to a reflecting surface 50b of the light transmitting layer 50 on which the coherent light is reflected,

D₂ is a distance from a light shielding surface 60c of the light shielding film 60 to the reflecting surface 50b described above,

D₃ is a distance from the light receiving surface of the light receiving part 30 to the reflecting surface 50b described above,

L₁ is a distance from a position corresponding to the light emitting center of the light emitting part 20 in the light emitting side opening part 60a to a proximal end of the light receiving side opening part 60b, and

L₂ is a distance from the light emitting center of the light emitting part 20 to a distal end of the light receiving part 30.

The light receiving side opening part 60b of the light shielding film 60 is configured to shield the reflected light E₃ that is from the interface of the light transmitting layer 50 on the basis of the Equation (1) described above.

FIG. 3 schematically shows a case where coherent light is reflected by the outer surface 50b of the light transmitting layer 50 in contact with the user's skin. In the present embodiment, a case where the reflecting surface of the light transmitting layer 50 on which coherent light is reflected in D₁, D₂, and D₃ of the Equation (1) described above is the outer surface 50b of the light transmitting layer 50 is shown. However, the reflecting surface of the light transmitting layer 50 on which coherent light is reflected is not limited to the outer surface 50b of the light transmitting layer 50. For example, in a case where it is assumed that the outer surface 50b of the light transmitting layer 50 is always in contact with the skin and does not move, it is preferable that the reflecting surface on which the coherent light is reflected is the inner surface 50a of the light transmitting layer 50. In this case, in the Equation (1) described above, D₁ is a distance from the light emitting center of the light emitting part 20 to the reflecting surface 50a of the light transmitting layer 50 on which the coherent light is reflected, D₂ is a distance from the light shielding surface 60c of the light shielding film 60 to the reflecting surface 50a, and D₃ is a distance from the light receiving surface of the light receiving part 30 to the reflecting surface 50a.

As described above, the reflecting surface of the light transmitting layer on which the coherent light is reflected in the Equation (1) described above means the inner surface or the outer surface of the light transmitting layer.

In the biometric information measuring apparatus 10 of the present embodiment, the light shielding film 60 is provided on the inner surface 41a of the lid part 41, but the position of the light shielding film 60 is not limited to this. The light shielding film 60 may be provided on the outer surface 41b of the lid part 41. Even in this case, the biometric information measuring apparatus 10 is configured to satisfy the Equation (1) described above. Furthermore, the light shielding film 60 may be provided on both the inner surface 41a and the outer surface 41b of the lid part 41. In this case, the biometric information measuring apparatus 10 is configured to satisfy the Equation (1) described above for each of the two light shielding films. From the viewpoint of simplification of the manufacturing process and cost reduction, it is preferable that one light shielding film 60 is provided and the light shielding film 60 is provided on the inner surface 41a of the lid part 41 or the outer surface 41b of the lid part 41.

The member included in the light shielding film 60 is not particularly limited as long as it blocks light, and may be a light absorbing member or a light reflecting member. That is, the light shielding film 60 may be a light reflecting film or a light absorbing film. The light shielding film 60 is preferably a conductive member formed by means such as metal vapor deposition, and is preferably grounded appropriately. Therefore, not only light but also unnecessary electromagnetic waves can be shielded, and electromagnetic noise generated by the invasion of unnecessary electromagnetic waves into the housing 40 can be suppressed.

In the present embodiment, a laser Doppler blood flow meter has been described as an example as an embodiment of the biometric information measuring apparatus according to the present technology. The laser Doppler blood flow meter can irradiate a human skin surface with a laser beam to measure blood flow in capillaries non-invasively and continuously, and is small in size. Therefore, the biometric information measuring apparatus 10 of the present embodiment is suitable for a wearable type blood flow meter such as a headband type, a neckband type, and a belt type. Examples of other embodiments of the biometric information measuring apparatus according to the present technology include any personal digital assistant (PDA) such as a smartphone or tablet terminal, and any electronic device such as a medical device, a game device, a home appliance device, or the like.

Examples of the biological information that can be measured by using the biometric information measuring apparatus of the present embodiment include blood flow rate, blood cell volume, blood flow velocity, pulse rate, and the like.

### 5. Second Embodiment

A biometric information measuring apparatus according to a second embodiment of the present technology will be described.

FIG. 4 is a schematic cross-sectional view showing a part of a biometric information measuring apparatus 11 according to a second embodiment. A light shielding film 61 of the biometric information measuring apparatus 11 according to the present embodiment has a different configuration from that of the light shielding film 60 of the biometric information measuring apparatus 10 according to the first embodiment described above. Hereinafter, the points different from the first embodiment described above will be mainly described.

The light shielding film 61 included in the biometric information measuring apparatus 11 is provided on an inner surface 41a of the lid part 41, and has a light emitting side opening part 61a facing the light emitting part 20 and a light receiving side opening part 61b facing the light receiving part 30. FIG. 4 shows a cross section of a surface passing through the light emitting center of the light emitting part 20, the light receiving surface center of the light receiving part 30, the center of the light emitting side opening part 61a, and the center of the light receiving side opening part 61b.

The light shielding film 61 of the biometric information measuring apparatus 11 of the present embodiment prevents the coherent light emitted from the light emitting part 20 from being reflected by the light transmitting layer 50. That is, the light shielding film 61 shields the coherent light and prevents the coherent light from reaching the light transmitting layer 50, thereby preventing the coherent light from being reflected by the light transmitting layer 50. Therefore, it is possible to reduce noise caused by the light reflected at the interface of the light transmitting layer 50 when a body movement occurs.

The biometric information measuring apparatus 11 of the present embodiment satisfies the Equation (2) described below in a case where D₁, D₂, D₃, L₂, and L₃ are defined as follows.

$$\begin{matrix}[ Math . 10 ] & \\ \frac{{L}_{3}}{{D}_{1} - {D}_{2}} \leq \frac{{L}_{2}}{{D}_{1} + {D}_{3}} & ( 2 )\end{matrix}$$

In the Equation (2) described above,

D₁ is a distance from the light emitting center of the light emitting part 20 to the reflecting surface 50b of the light transmitting layer 50 on which the coherent light can be reflected,

D₂ is a distance from the light shielding surface 61c of the light shielding film 61 to the reflecting surface 50b described above,

D₃ is a distance from the light receiving surface of the light receiving part 30 to the reflecting surface 50b described above,

L₂ is a distance from the light emitting center of the light emitting part 20 to a distal end of the light receiving part 30, and

L₃ is a distance from a position corresponding to the light emitting center of the light emitting part 20 in the light emitting side opening part 61a to an end of a center direction of the light receiving side opening part 61b of the light emitting side opening part 61a.

In the example shown in FIG. 4, in a case where the coherent light emitted from the light emitting part 20 is not shielded by the light shielding surface (face) 61c of the light shielding film 61, the coherent light is reflected by the outer surface 50b of the light transmitting layer 50, and reflected light is generated from the outer surface 50b toward the light receiving part 30. Therefore, the light shielding film 61 of the biometric information measuring apparatus 11 of the present embodiment shields coherent light E₄ emitted from the light emitting part 20 with the light shielding surface 61c, thereby preventing the coherent light E₄ from reaching the light transmitting layer 50. Therefore, coherent light is prevented from being reflected by the reflecting surface (outer surface) 50b of the light transmitting layer 50. The light emitting side opening part 61a of the light shielding film 61 is configured to shield the coherent light E₄ on the basis of the Equation (2) described above.

In the present embodiment, a case where the reflecting surface of the light transmitting layer 50 on which coherent light can be reflected in D₁, D₂, and D₃ of the Equation (2) described above is the outer surface 50b of the light transmitting layer 50 is shown. However, the reflecting surface of the light transmitting layer 50 on which coherent light can be reflected is not limited to the outer surface 50b of the light transmitting layer 50. For example, in a case where it is assumed that the outer surface 50b of the light transmitting layer 50 is always in contact with the skin and does not move, it is preferable that the reflecting surface on which the coherent light can be reflected is the inner surface 50a of the light transmitting layer 50. In this case, in the Equation (2) described above, D₁ is a distance from the light emitting center of the light emitting part 20 to the reflecting surface 50a of the light transmitting layer 50 on which the coherent light can be reflected, D₂ is a distance from the light shielding surface 61c of the light shielding film 61 to the reflecting surface 50a, and D₃ is a distance from the light receiving surface of the light receiving part 30 to the reflecting surface 50a.

As described above, the reflecting surface of the light transmitting layer on which the coherent light can be reflected in the Equation (2) described above means the inner surface or the outer surface of the light transmitting layer.

### 6. Third Embodiment

A biometric information measuring apparatus according to a third embodiment of the present technology will be described.

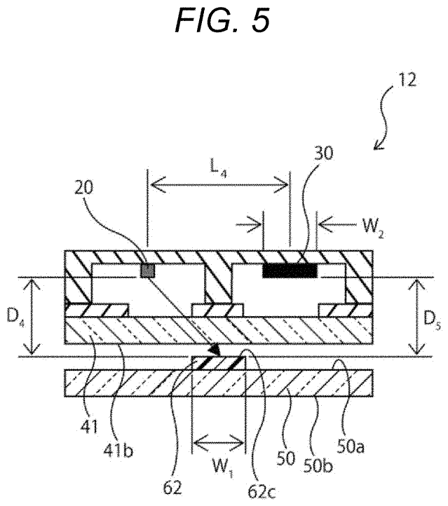

FIG. 5 is a schematic cross-sectional view showing a part of a biometric information measuring apparatus 12 according to a third embodiment. A light shielding film 62 of the biometric information measuring apparatus 12 according to the present embodiment has a different configuration from that of the light shielding film 60 of the biometric information measuring apparatus 10 according to the first embodiment described above. Hereinafter, the points different from the first embodiment described above will be mainly described.

The light shielding film 62 included in the biometric information measuring apparatus 12 is provided on the inner surface 50a of the light transmitting layer 50. FIG. 5 shows a cross section of a surface passing through the light emitting center of the light emitting part 20, the light receiving surface center of the light receiving part 30, and the center of the light shielding film 62.

The light shielding film 62 of the biometric information measuring apparatus 12 of the present embodiment prevents the coherent light emitted from the light emitting part 20 from being reflected by the light transmitting layer 50. That is, the light shielding film 62 shields the coherent light with the light shielding surface 62c and prevents the coherent light from reaching the light transmitting layer 50, thereby preventing the coherent light from being reflected by the light transmitting layer 50. Therefore, it is possible to reduce noise caused by the light reflected at the interface of the light transmitting layer 50 when a body movement occurs.

The biometric information measuring apparatus 12 of the present embodiment satisfies the Equation (3) described below in a case where D₄, D₅, L₄, W₁, and W₂ are defined as follows.

$$\begin{matrix}[ Math . 11 ] & \\ {W}_{1} \geq \frac{{D}_{4} - {D}_{5}}{{D}_{4} + {D}_{5}} {L}_{4} + \frac{{D}_{4}}{{D}_{4} + {D}_{5}} {W}_{2} & ( 3 )\end{matrix}$$

In the Equation (3) described above,

D₄ is a distance from a light emitting center of the light emitting part 20 to a plane including a light shielding surface 62c of the light shielding film 62,

D₅ is a distance from a light receiving surface of the light receiving part 30 to the plane including the light shielding surface 62c of the light shielding film 62,

L₄ is a distance from the light emitting center of the light emitting part 20 to a light receiving surface center of the light receiving part 30,

W₁ is a length of the light shielding film 62 in a width direction when a direction connecting the light emitting center of the light emitting part 20 and the light receiving surface center of the light receiving part 30 is the width direction, and

W₂ is a length of the light receiving part 30 in the width direction.

In a case where the D₄ and D₅ described above can be regarded as almost equal, the relationship between W₁ and W₂ is expressed by the Equation (3′) described below.

$$\begin{matrix}[ Math . 12 ] & \\ {W}_{1} \geq \frac{1}{2} {W}_{2} & ( {3}^{′} )\end{matrix}$$

In the biometric information measuring apparatus 12 of the present embodiment, the light shielding film 62 is provided on the inner surface 50a of the light transmitting layer 50, but the position of the light shielding film 62 is not limited to this. The light shielding film 62 is only required to be provided on at least one of the outer surface 41b of the lid part 41, the inner surface 50a of the light transmitting layer 50, or the outer surface 50b of the light transmitting layer 50. In a case where a plurality of light shielding films 62 is provided, each of the plurality of light shielding films 62 is configured to satisfy the Equation (3) described above. From the viewpoint of ease of manufacture and cost reduction, a configuration including one light shielding film 62 is preferable. From the viewpoint of ease of manufacture and prevention of adhesion of dirt, the light shielding film 62 is preferably provided on the inner surface 50a of the light transmitting layer 50.

The member forming the light shielding film 62 is preferably a member other than a light reflecting member, and preferably a light absorbing member. That is, the light shielding film 62 is preferably a film other than a light reflecting film, and is preferably a light absorbing film.

In the above-mentioned first to third embodiments, the Equations (1) to (3′) described above define the minimum necessary requirements for preventing reflected light, and in an actual manufacturing process, a configuration is adopted in which attaching tolerance and mounting tolerance of the light transmitting layer 50 are taken into consideration, for example.

### 7. Fourth Embodiment

A biometric information measuring apparatus according to a fourth embodiment of the present technology will be described.

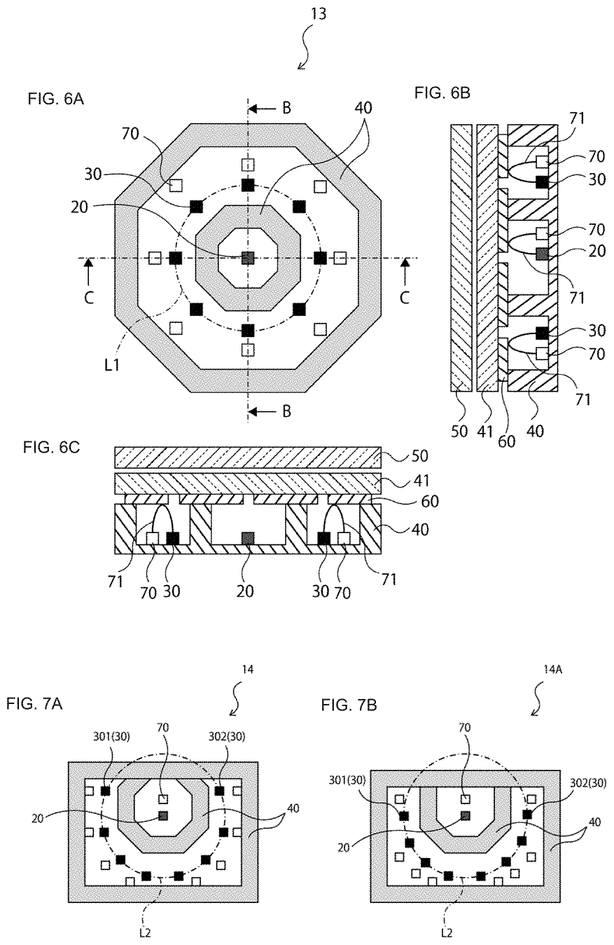

FIG. 6 is a schematic view showing a part of a biometric information measuring apparatus 13 according to a fourth embodiment. FIG. 6A is a plan view of a part of the biometric information measuring apparatus 13. FIG. 6B is a cross-sectional view taken along the line B-B of the biometric information measuring apparatus 13 shown in FIG. 6A. FIG. 6C is a cross-sectional view taken along the line C-C of the biometric information measuring apparatus 13 shown in FIG. 6A. In FIG. 6A, the light shielding film 60, the lid part 41, and the light transmitting layer 50 shown in FIGS. 6B and 6C are not shown. The housing 40 of the biometric information measuring apparatus 13 according to the present embodiment accommodates two or more light receiving parts 30 therein. Hereinafter, the points different from the first embodiment described above will be mainly described.

In the biometric information measuring apparatus 13 shown in FIG. 6, eight light receiving parts 30 are accommodated in the housing 40. By providing two or more light receiving parts 30 as described above, it is possible to improve the light detection accuracy.

As shown in FIG. 6A, the eight light receiving parts 30 are arranged at equal intervals on the concentric circles indicated by the virtual line L1 in a plan view. As shown in FIGS. 6B and 6C, the light emitting part 20 and the light receiving part 30 are each connected to a connection pad 70 by a bonding wire 71. Each component shown in FIGS. 6B and 6C is configured to satisfy the Equation (1) as similar to the first embodiment described above.

The biometric information measuring apparatus 13 of the present embodiment includes a light shielding film that is similar to the light shielding film 60 of the first embodiment described above, but the configuration of the light shielding film is not limited to this. The light shielding film may be a light shielding film that is similar to that of the second embodiment or the third embodiment described above.

### 8. Fifth Embodiment

A biometric information measuring apparatus according to a fifth embodiment of the present technology will be described.

FIG. 7 is a schematic plan view showing a part of a biometric information measuring apparatus 14 according to a fifth embodiment. FIG. 7A shows the biometric information measuring apparatus 14 according to the fifth embodiment, and FIG. 7B shows a biometric information measuring apparatus 14A according to a modification of the fifth embodiment. The housing 40 of the biometric information measuring apparatus 14 according to the present embodiment accommodates therein three or more light receiving parts 30, and the light receiving parts 30 are arranged at unequal intervals. Hereinafter, the points different from the fourth embodiment described above will be mainly described.

As shown in FIGS. 7A and 7B, the eight light receiving parts 30 are arranged at unequal intervals on the concentric circles indicated by the virtual line L2 in a plan view. A connection pat 70 that is electrically connected to the light emitting part 20 is provided at a position corresponding to a portion between the light receiving parts 30 and specifically, a position corresponding to a portion between the light receiving part 301 and the light receiving part 302. The interval between the light receiving part 301 and the light receiving part 302 is larger than the interval between the other light receiving parts 30. As described above, by arranging three or more light receiving parts 30 at an unequal intervals, and making at least one interval be larger than the other intervals, the connection pad 70 of the light emitting part 20 can be arranged in the portion where the interval is large. Therefore, the outer shape of the housing 40 can be kept small.

### 9. Sixth Embodiment

A biometric information measuring apparatus according to a sixth embodiment of the present technology will be described.

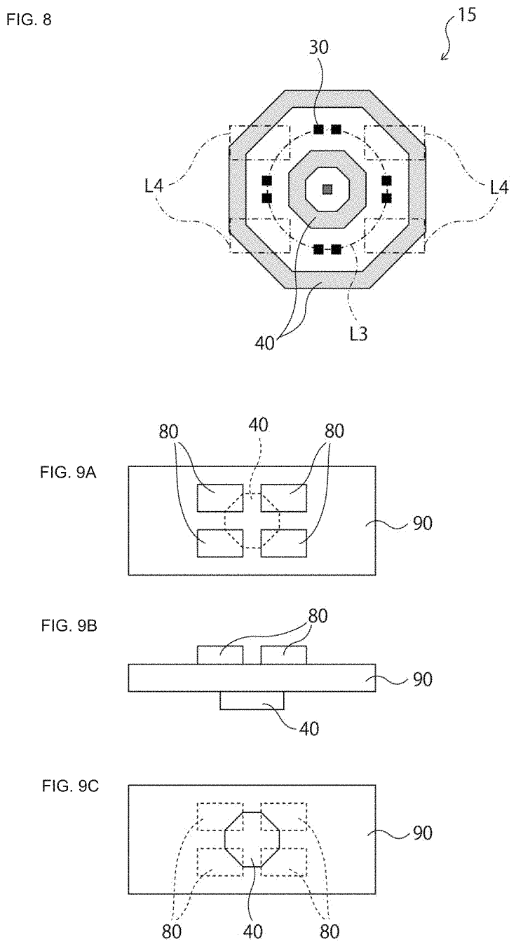

FIG. 8 is a schematic plan view showing a part of a biometric information measuring apparatus 15 according to a sixth embodiment. The housing 40 of the biometric information measuring apparatus 15 according to the present embodiment accommodates eight light receiving parts 30 therein, and an electrical component can be provided at a position corresponding to a portion between the light receiving parts 30. Hereinafter, the points different from the fourth embodiment described above will be mainly described.

As shown in FIG. 8, the eight light receiving parts 30 are arranged at equal intervals on the concentric circles indicated by the virtual line L3 in a plan view. In addition, the eight light receiving parts 30 are arranged in four groups of two each. By arranging the light receiving parts 30 in a plurality of groups as described above, it is possible to provide a space for arranging an electrical component between the light receiving parts 30 indicated by the virtual line L4. For example, as an electrical component, a semiconductor circuit (operational amplifier) that amplifies the output of the light receiving part 30 can be arranged. By adopting the configuration of the present embodiment, the distance between the light receiving part 30 and the semiconductor circuit can be minimized, so that a noise-resistant biometric information measuring apparatus can be manufactured.

FIG. 9 is a schematic view showing a substrate 90 on which a housing 40 shown in FIG. 8 is mounted. FIG. 9A is a plan view of the substrate 90 on which the housing 40 shown in FIG. 8 is mounted, FIG. 9B is a side view of FIG. 9A, and FIG. 9C is a bottom view of FIG. 9A. As shown in FIG. 9, the housing 40 is mounted on the lower surface of the substrate 90. Furthermore, four electrical components 80 are mounted on the upper surface of the substrate 90. The housing 40 corresponds to a blood flow sensor, and the electrical component 80 is, for example, an operational amplifier.

Note that the fifth and sixth embodiments described above are illustrated as flip-chip bonding in which the light emitting part and the light receiving part are mounted on electrodes without having a power supply provided at different positions on a plane, but the mounting method is not limited to this, and may be, for example, wire bonding.

It is possible to combine two or more feature parts among the feature parts related to the present technology described above. That is, the various feature portions described in the embodiments may be arbitrarily combined without distinction among the embodiments.

Note that, the present technology can also adopt the following configuration.

[1] A biometric information measuring apparatus including:

a housing that accommodates therein a light emitting part that emits coherent light and a light receiving part and has a lid part through which the coherent light transmits;

a light transmitting layer located outside the lid part and through which the coherent light transmits; and

a light shielding film that prevents the coherent light from being reflected by the light transmitting layer and reaching the light receiving part.

[2] The biometric information measuring apparatus according to [1],

in which the light shielding film is provided on at least one of an inner surface of the lid part or an outer surface of the lid part, and has a light emitting side opening part facing the light emitting part and a light receiving side opening part facing the light receiving part, and

in a case where a distance from a light emitting center of the light emitting part to a reflecting surface of the light transmitting layer on which the coherent light is reflected is D₁,

a distance from a light shielding surface of the light shielding film to the reflecting surface is D₂,

a distance from a light receiving surface of the light receiving part to the reflecting surface is D₃,

a distance from a position corresponding to the light emitting center of the light emitting part in the light emitting side opening part to a proximal end of the light receiving side opening part is L₁, and

a distance from the light emitting center of the light emitting part to a distal end of the light receiving part is L₂,

the Equation (1) described below is satisfied:

$$\begin{matrix}[ Math . 13 ] & \\ \frac{{L}_{1}}{{D}_{1} + {D}_{2}} \geq \frac{{L}_{2}}{{D}_{1} + {D}_{3}} & ( 1 )\end{matrix}$$

[3] The biometric information measuring apparatus according to [1] or [2],

in which the light shielding film is provided on at least one of an inner surface of the lid part or an outer surface of the lid part, and has a light emitting side opening part facing the light emitting part and a light receiving side opening part facing the light receiving part, and

in a case where a distance from a light emitting center of the light emitting part to a reflecting surface of the light transmitting layer on which the coherent light can be reflected is D₁,

a distance from a light shielding surface of the light shielding film to the reflecting surface is D₂,

a distance from a light receiving surface of the light receiving part to the reflecting surface is D₃,

a distance from the light emitting center of the light emitting part to a distal end of the light receiving part is L₂, and

a distance from a position corresponding to the light emitting center of the light emitting part in the light emitting side opening part to an end of a center direction of the light receiving side opening part of the light emitting side opening part is L₃,

the Equation (2) described below is satisfied:

$$\begin{matrix}[ Math . 14 ] & \\ \frac{{L}_{3}}{{D}_{1} - {D}_{2}} \leq \frac{{L}_{2}}{{D}_{1} + {D}_{3}} & ( 2 )\end{matrix}$$

[4] The biometric information measuring apparatus according to any one of [1] to [3],

in which the light shielding film is provided on at least one of an outer surface of the lid part, an inner surface of the light transmitting layer, or an outer surface of the light transmitting layer, and

in a case where a distance from a light emitting center of the light emitting part to a plane including a light shielding surface of the light shielding film is D₄,

a distance from a light receiving surface of the light receiving part to the plane including the light shielding surface of the light shielding film is D₅,

a distance from the light emitting center of the light emitting part to a light receiving surface center of the light receiving part is L₄,

a length of the light shielding film in a width direction when a direction connecting the light emitting center of the light emitting part and the light receiving surface center of the light receiving part is the width direction is W₁, and

a length of the light receiving part in the width direction is W₂,

the Equation (3) described below is satisfied:

$$\begin{matrix}[ Math . 15 ] & \\ {W}_{1} \geq \frac{{D}_{4} - {D}_{5}}{{D}_{4} + {D}_{5}} {L}_{4} + \frac{{D}_{4}}{{D}_{4} + {D}_{5}} {W}_{2} & ( 3 )\end{matrix}$$

[5] The biometric information measuring apparatus according to any one of [1] to [4], in which the housing contains two or more of the light receiving part therein.
[6] The biometric information measuring apparatus according to any one of [1] to [5],

in which the housing accommodates three or more of the light receiving part therein, and

the three or more of the light receiving part are arranged at unequal intervals.

[7] The biometric information measuring apparatus according to [6], further including a connection pad electrically connected to the light emitting part at a position corresponding to a portion between the three or more of the light receiving part.
[8] The biometric information measuring apparatus according to [6] or [7], further including an electrical component at a position corresponding to a portion between the three or more of the light receiving part.

### REFERENCE SIGNS LIST

10, 11, 12, 13, 14, 15 Biometric information measuring apparatus
20 Light emitting part
30 Light receiving part
40 Housing
41 Lid part
50 Light transmitting layer
60, 61, 62 Light shielding film
70 Connection pad
71 Bonding wire
80 Electronic component
90 Substrate
100 Main body part

1. A biometric information measuring apparatus comprising:
a housing that accommodates therein a light emitting part that emits coherent light and a light receiving part and has a lid part through which the coherent light transmits;
a light transmitting layer located outside the lid part and through which the coherent light transmits; and
a light shielding film that prevents the coherent light from being reflected by the light transmitting layer and reaching the light receiving part.

2. The biometric information measuring apparatus according to claim 1,
wherein the light shielding film is provided on at least one of an inner surface of the lid part or an outer surface of the lid part, and has a light emitting side opening part facing the light emitting part and a light receiving side opening part facing the light receiving part, and
in a case where a distance from a light emitting center of the light emitting part to a reflecting surface of the light transmitting layer on which the coherent light is reflected is D₁,
a distance from a light shielding surface of the light shielding film to the reflecting surface is D₂,
a distance from a light receiving surface of the light receiving part to the reflecting surface is D₃,
a distance from a position corresponding to the light emitting center of the light emitting part in the light emitting side opening part to a proximal end of the light receiving side opening part is L₁, and
a distance from the light emitting center of the light emitting part to a distal end of the light receiving part is L₂,
the Equation (1) described below is satisfied:
$$\begin{matrix}[ Math . 1 ] & \\ \frac{{L}_{1}}{{D}_{1} + {D}_{2}} \geq \frac{{L}_{2}}{{D}_{1} + {D}_{3}} & ( 1 )\end{matrix}$$

3. The biometric information measuring apparatus according to claim 1,
wherein the light shielding film is provided on at least one of an inner surface of the lid part or an outer surface of the lid part, and has a light emitting side opening part facing the light emitting part and a light receiving side opening part facing the light receiving part, and
in a case where a distance from a light emitting center of the light emitting part to a reflecting surface of the light transmitting layer on which the coherent light can be reflected is D₁,
a distance from a light shielding surface of the light shielding film to the reflecting surface is D₂,
a distance from a light receiving surface of the light receiving part to the reflecting surface is D₃,
a distance from the light emitting center of the light emitting part to a distal end of the light receiving part is L₂, and
a distance from a position corresponding to the light emitting center of the light emitting part in the light emitting side opening part to an end of a center direction of the light receiving side opening part of the light emitting side opening part is L₃,
the Equation (2) described below is satisfied:
$$\begin{matrix}[ Math . 2 ] & \\ \frac{{L}_{3}}{{D}_{1} - {D}_{2}} \leq \frac{{L}_{2}}{{D}_{1} + {D}_{3}} & ( 2 )\end{matrix}$$

4. The biometric information measuring apparatus according to claim 1,
wherein the light shielding film is provided on at least one of an outer surface of the lid part, an inner surface of the light transmitting layer, or an outer surface of the light transmitting layer, and
in a case where a distance from a light emitting center of the light emitting part to a plane including a light shielding surface of the light shielding film is D₄,
a distance from a light receiving surface of the light receiving part to the plane including the light shielding surface of the light shielding film is D₅,
a distance from the light emitting center of the light emitting part to a light receiving surface center of the light receiving part is L₄,
a length of the light shielding film in a width direction when a direction connecting the light emitting center of the light emitting part and the light receiving surface center of the light receiving part is the width direction is W₁, and
a length of the light receiving part in the width direction is W₂,
the Equation (3) described below is satisfied:
$$\begin{matrix}[ Math . 3 ] & \\ {W}_{1} \geq \frac{{D}_{4} - {D}_{5}}{{D}_{4} + {D}_{5}} {L}_{4} + \frac{{D}_{4}}{{D}_{4} + {D}_{5}} {W}_{2} & ( 3 )\end{matrix}$$

5. The biometric information measuring apparatus according to claim 1, wherein the housing contains two or more of the light receiving part therein.

6. The biometric information measuring apparatus according to claim 1,
wherein the housing accommodates three or more of the light receiving part therein, and
the three or more of the light receiving part are arranged at unequal intervals.

7. The biometric information measuring apparatus according to claim 6, further comprising a connection pad electrically connected to the light emitting part at a position corresponding to a portion between the three or more of the light receiving part.

8. The biometric information measuring apparatus according to claim 6, further comprising an electrical component at a position corresponding to a portion between the three or more of the light receiving part.
